(19) United States
(12) Patent Application Publication
(10) Pub. No.: US 2013/0324782 A1
(43) Pub. Date: Dec. 5, 2013

(54) METHOD FOR ISOMERIZATION OF PARAFFIN HYDROCARBONS C4-C7

(71) Applicant: JOINT STOCK COMPANY SCIENTIFIC INDUSTRIAL ENTERPRI, Krasnodar (RU)

(72) Inventors: Alexander Nikitovich SHAKUN, Krasnodar (RU); Marina Leonidovna FEDOROVA, Krasnodar (RU)

(73) Assignee: Joint Stock Company Scientific Industrial Enterprise Neftehim (JSC SIE Nefthim), Krasnodar (RU)

(21) Appl. No.: 13/682,392

(22) Filed: Nov. 20, 2012

(30) Foreign Application Priority Data
May 29, 2012 (RU) 2012122289

(51) Int. Cl. C07C 5/22

(52) U.S. Cl. CPC C07C 5/2213
USPC 585/751

(57) ABSTRACT

The method for isomerization of paraffin hydrocarbons C₄-C₇ for production of high-octane gasoline components is disclosed, it can be used in the oil refining and petrochemical industries. Paraffin hydrocarbons C₄-C₇ are isomerized on a porous zirconium oxide catalyst with the average pore diameter within 8 to 24 nm in a hydrogen atmosphere at the temperature of 100-250° C. and pressure of 1.0-5.0 MPa, molar ratio H₂:hydrocarbons of (0.1-5):1, feed space velocity of 0.5-6.0 h⁻¹ and under isomerate stabilization and/or fractionation with recovery of individual hydrocarbons or high-octane fractions. Zirconium oxide catalyst has the following composition, weight %: 97.00-99.90 of a carrier, the carrier comprising: zirconium oxide (60.00-86.00), aluminum oxide (10.00-30.00), titanium oxide (0.05-2.00), manganese oxide (0.05-2.00), iron oxide (0.05-2.00), SO₄²⁻ or WO₃²⁻ (3.00-20.00).

### RELATED APPLICATIONS

This application claims priority to Russian Patent Application No. 2012122289, filed May 29, 2012, which is incorporated herein by reference in its entirety.

### FIELD OF THE INVENTION

The invention pertains to the method for isomerization of paraffin hydrocarbons C₄-C₇ for production of high-octane gasoline components and can be used in the oil refining and petrochemical industries.

### BACKGROUND OF THE INVENTION

The closest approach to the present invention in terms of technical substance is the U.S. Pat. No. 6,495,733 B01 J 27/053 Superacid catalyst for hydroisomerization of n-paraffins. According to this invention, a porous zirconium oxide catalyst, in which not less than 70% of pores have a diameter of 1-4 nm, is used in isomerization of n-paraffin hydrocarbons.

The disadvantage of this isomerization method is the low process stability and incomplete recoverability of the catalyst activity after regeneration. Thus, when implementing the process of C₅-C₆ paraffin hydrocarbons isomerization according to U.S. Pat. No. 6,495,733 using a catalyst, in which 75% of pores with the diameter from 1 to 4 nm, at the temperature of 150° C., pressure of 3.0 MPa, feed space velocity of 3 h⁻¹, and molar ratio hydrogen:feedstock of 2:1, the catalyst activity in isomerization of C₅-C₆ is reduced by 10% after 200 hours.

### SUMMARY OF THE INVENTION

Paraffin hydrocarbons C₄-C₇ are isomerized on a porous zirconium oxide catalyst with the average pore diameter within 8 to 24 nm in a hydrogen atmosphere at the temperature of 100-250° C. and pressure of 1.0-5.0 MPa, molar ratio H₂:hydrocarbons of (0.1-5):1, feed space velocity of 0.5-6.0 h⁻¹. Products of isomerization are stabilized and/or fractioned to recover individual hydrocarbons or high-octane fractions.

### DETAILED DESCRIPTION OF THE PREFERRED EMBODIMENTS

Method for isomerization of light paraffin hydrocarbons is implemented as follows.

N-butane, C₅-C₆ cut or C₇ cut are used as a feedstock.

The feedstock composition is given in Table 1.

The feedstock is mixed with hydrogen or hydrogen-bearing gas (HBG), heated to the temperature of 100-250° C., pressure of 1.0-5.0 MPa, molar ratio H₂:hydrocarbons of (0.1-5):1, and feed space velocity of 0.5-6.0 hour⁻¹, and fed to a reactor filled with a porous catalyst with the average pore diameter from 8 to 24 nm, which contains 0.1-3.0 weight % of a hydrogenating component on a carrier, consisting of sulfated and/or tungstated zirconium, aluminum, titanium, manganese, and iron oxides.

The reaction product is analyzed by gas-liquid chromatography using a capillary column with the OV-1 phase applied.

The isomerization depth is determined:
During isomerization of n-butane on the basis of n-butane conversion, %;
During isomerization of C₅-C₆ cut on the basis of concentration of the most branched isomer of 2.2-dimethylbutane in the amount of all C₆H₁₄ isomers;
During isomerization of C₇ cut on the basis of concentration of di- and tri-substituted C₇ isomers in the amount of all C₇H₁₆ isomers.

The proposed method offers the stable isomerization depth of unbranched paraffin hydrocarbons C₄-C₇ during the entire service cycle and after its regeneration.

Sulfated or tungstated zirconium dioxide in combination with aluminum oxide, titanium oxide, manganese oxide, and iron oxide is used as the catalyst carrier for isomerization of paraffin hydrocarbons C₄-C₇. The hydrogenating component is selected from platinum, palladium, nickel, gallium, or zinc metals.

The carrier for the catalyst of normal paraffins isomerization is prepared by mixing the components followed by extruding, drying, and calcination at 500-800° C. The catalyst is prepared by impregnating the carrier with a solution containing the hydrogenating component and subsequent drying and calcination at 400-550° C. in the air flow. The average diameter of pores of the resultant catalyst is determined by the BET method.

The process efficiency depends on the maintenance of a constant isomerization depth during operation and after regeneration of the catalyst.

Coke is deposited on the catalyst surface during operation. Some active sites become inaccessible for the source hydrocarbon as the surface deposits built up, which results in reduction of the isomerization depth. The catalyst activity is recovered by regeneration, which consists in high-temperature treatment of the catalyst in the nitrogen flow, containing 1-10 vol. % of oxygen.

Presence of nano-pores with the radius of 8-24 nm is a prerequisite for maintaining the constant isomerization depth in operation and after oxidative regeneration. The use of a catalyst with smaller pores (below 8 nm) results in reduction of the isomerization depth in the course of operation and it is incompletely recovered after oxidative regeneration. The use of a catalyst with larger pores (over 24 nm) results in reduction of the isomerization depth.

### Example 1

N-butane is used as the feedstock. The process is implemented on a pilot plant at the temperature of 180° C., pressure of 1.0 MPa, molar ratio H₂:hydrocarbon of 0.1:1 and feed space velocity of 1.0 h⁻¹ on a catalyst with the average pore diameter of 8 nm, which has the following composition, weight %:

| | |
|---|---|
| Zirconium oxide | 71.81 |
| Aluminum oxide | 15.00 |
| Titanium oxide | 0.05 |
| Manganese oxide | 0.05 |
| Iron oxide | 0.09 |
| Sulfuric acid ion SO₄²⁻ | 12.00 |

1.0% Ga is used as the hydrogenating component.

Composition of the n-butane isomerization feedstock is given in Table 1.

Depth of n-butane isomerization into isobutane after 10, 200 hours and after regeneration of the catalyst is given in Table 2.

The catalyst is coked after 200 hours of continuous operation. To do this, the molar ratio hydrogen:hydrocarbons is set to 0.02:1, the temperature raised to 250° C. and held for 20 hours. After coking, the regeneration at the temperature of 500° C. in the nitrogen flow with 5 vol. % of oxygen is performed. Upon completion of regeneration, the experiment is conducted under the previous conditions.

### Example 2

Isomerization is performed according to the method of example 1 differing in that:
The process is implemented on a catalyst with the average pore diameter of 24 nm, which has the following composition, weight %:

| | |
|---|---|
| Zirconium oxide | 63.91 |
| Aluminum oxide | 28.00 |
| Titanium oxide | 1.00 |
| Manganese oxide | 0.90 |
| Iron oxide | 0.19 |
| Sulfuric acid ion SO₄²⁻ | 3.00 |

3.0% Ga is used as the hydrogenating component. The process is implemented at the temperature of 180° C., pressure of 2.0 MPa, molar ratio H₂:hydrocarbon of 1.0:1, and feed space velocity of 6.0 h⁻¹.

Depth of n-butane isomerization into isobutane after 10, 200 hours and after regeneration of the catalyst is given in Table 2.

### Example 3

Isomerization is performed according to the method of example 1 differing in that:
The process is implemented on a catalyst with the average pore diameter of 22 nm, which has the following composition, weight %:

| | |
|---|---|
| Zirconium oxide | 60.00 |
| Aluminum oxide | 16.00 |
| Titanium oxide | 0.10 |
| Manganese oxide | 0.70 |
| Iron oxide | 2.00 |
| Sulfuric acid ion SO₄²⁻ | 20.00 |

Zn in the amount of 1.2% is used as the hydrogenating component. The process is implemented at the temperature of 200° C., pressure of 1.0 MPa, molar ratio H₂:hydrocarbon of 1.0:1, and feed space velocity of 2.0 h⁻¹.

Depth of n-butane isomerization into isobutane after 10, 200 hours and after regeneration of the catalyst is given in Table 2.

### Example 4

Isomerization is performed according to the method of example 1 differing in that:
The process is implemented on a catalyst with the average pore diameter of 20 nm, which has the following composition, weight %:

| | |
|---|---|
| Zirconium oxide | 63.66 |
| Aluminum oxide | 22.00 |
| Titanium oxide | 1.50 |
| Manganese oxide | 1.50 |
| Iron oxide | 0.54 |
| Sulfuric acid ion SO₄²⁻ | 8.00 |

Zn in the amount of 2.8% is used as the hydrogenating component. The process is implemented at the temperature of 220° C., pressure of 2.0 MPa, molar ratio H₂:hydrocarbon of 1.0:1, and feed space velocity of 4.0 h⁻¹.

Depth of n-butane isomerization into isobutane after 10, 200 hours and after regeneration of the catalyst is given in Table 2.

### Example 5

Isomerization is performed according to the method of example 1 differing in that:
The process is implemented on a catalyst with the average pore diameter of 20 nm, which has the following composition, weight %:

| | |
|---|---|
| Zirconium oxide | 63.55 |
| Aluminum oxide | 18.00 |
| Titanium oxide | 2.00 |
| Manganese oxide | 1.90 |
| Iron oxide | 1.15 |
| Sulfuric acid ion SO₄²⁻ | 12.00 |

Ni in the amount of 1.4% is used as the hydrogenating component. The process is implemented at the temperature of 220° C., pressure of 1.0 MPa, molar ratio H₂:hydrocarbon of 1.0:1, and feed space velocity of 1.0 h⁻¹.

Depth of n-butane isomerization into isobutane after 10, 200 hours and after regeneration of the catalyst is given in Table 2.

### Example 6

Isomerization is performed according to the method of example 1 differing in that:
The process is implemented on a catalyst with the average pore diameter of 20 nm, which has the following composition, weight %:

| | |
|---|---|
| Zirconium oxide | 64.48 |
| Aluminum oxide | 17.00 |
| Titanium oxide | 1.40 |
| Manganese oxide | 1.60 |
| Iron oxide | 1.02 |
| Sulfuric acid ion SO₄²⁻ | 12.00 |

Ni in the amount of 2.5% is used as the hydrogenating component. The process is implemented at the temperature of 220° C., pressure of 1.5 MPa, molar ratio H₂:hydrocarbon of 3.0:1, and feed space velocity of 1.0 h⁻¹.

Depth of n-butane isomerization into isobutane after 10, 200 hours and after regeneration of the catalyst is given in Table 2.

### Example 7

### Comparative

Isomerization is performed according to the method of example 1 differing in that:
The process is implemented on a catalyst with the average pore diameter of 7 nm, which has the following composition, weight %:

| | |
|---|---|
| Zirconium oxide | 61.75 |
| Aluminum oxide | 26.00 |
| Titanium oxide | 0.05 |
| Manganese oxide | 0.05 |
| Iron oxide | 0.95 |
| Sulfuric acid ion SO₄²⁻ | 10.00 |

1.2% Ga is used as the hydrogenating component. The process is implemented at the temperature of 180° C., pressure of 1.0 MPa, molar ratio H₂:hydrocarbon of 1.0:1, and feed space velocity of 1.0 h⁻¹.

Depth of n-butane isomerization into isobutane after 10, 200 hours and after regeneration of the catalyst is given in Table 2.

### Example 8

### Comparative

Isomerization is performed according to the method of example 2 differing in that:
The process is implemented on a catalyst with the average pore diameter of 26 nm, which has the following composition, weight %:

| | |
|---|---|
| Zirconium oxide | 58.90 |
| Aluminum oxide | 30.00 |
| Titanium oxide | 1.00 |
| Manganese oxide | 1.00 |
| Iron oxide | 1.30 |
| Sulfuric acid ion SO₄²⁻ | 5.00 |

2.3% Ga is used as the hydrogenating component. The process is implemented at the temperature of 180° C., pressure of 2.0 MPa, molar ratio H₂:hydrocarbon of 1.0:1, and feed space velocity of 6.0 h⁻¹.

Depth of n-butane isomerization into isobutane after 10, 200 hours and after regeneration of the catalyst is given in Table 2.

### Example 9

### Comparative

Isomerization is performed according to the method of example 3 differing in that:
The process is implemented on a catalyst with the average pore diameter of 7 nm, which has the following composition, weight %:

| | |
|---|---|
| Zirconium oxide | 63.65 |
| Aluminum oxide | 12.00 |
| Titanium oxide | 1.15 |
| Manganese oxide | 0.40 |
| Iron oxide | 1.50 |
| Sulfuric acid ion SO₄²⁻ | 20.00 |

Zn in the amount of 1.3% is used as the hydrogenating component. The process is implemented at the temperature of 200° C., pressure of 1.0 MPa, molar ratio H₂:hydrocarbon of 1.0:1, and feed space velocity of 2.0 h⁻¹.

Depth of n-butane isomerization into isobutane after 10, 200 hours and after regeneration of the catalyst is given in Table 2.

### Example 10

### Comparative

Isomerization is performed according to the method of example 4 differing in that:
The process is implemented on a catalyst with the average pore diameter of 26 nm, which has the following composition, weight %:

| | |
|---|---|
| Zirconium oxide | 66.00 |
| Aluminum oxide | 10.00 |
| Titanium oxide | 1.00 |
| Manganese oxide | 1.20 |
| Iron oxide | 1.20 |
| Sulfuric acid ion SO₄²⁻ | 18.00 |

Zn in the amount of 2.6% is used as the hydrogenating component. The process is implemented at the temperature of 220° C., pressure of 2.0 MPa, molar ratio H₂:hydrocarbon of 1.0:1, and feed space velocity of 4.0 h⁻¹.

Depth of n-butane isomerization into isobutane after 10, 200 hours and after regeneration of the catalyst is given in Table 2.

### Example 11

### Comparative

Isomerization is performed according to the method of example 5 differing in that:
The process is implemented on a catalyst with the average pore diameter of 7 nm, which has the following composition, weight %:

| | |
|---|---|
| Zirconium oxide | 67.40 |
| Aluminum oxide | 15.00 |
| Titanium oxide | 1.50 |
| Manganese oxide | 1.40 |
| Iron oxide | 1.20 |
| Sulfuric acid ion SO₄²⁻ | 12.00 |

Ni in the amount of 1.5% is used as the hydrogenating component. The process is implemented at the temperature of 220° C., pressure of 1.0 MPa, molar ratio H₂:hydrocarbon of 1.0:1, and feed space velocity of 1.0 h⁻¹.

Depth of n-butane isomerization into isobutane after 10, 200 hours and after regeneration of the catalyst is given in Table 2.

### Example 12

### Comparative

Isomerization is performed according to the method of example 6 differing in that:
The process is implemented on a catalyst with the average pore diameter of 26 nm, which has the following composition, weight %:

| | |
|---|---|
| Zirconium oxide | 66.84 |
| Aluminum oxide | 18.00 |
| Titanium oxide | 0.07 |
| Manganese oxide | 0.09 |
| Iron oxide | 1.00 |
| Sulfuric acid ion SO₄²⁻ | 12.00 |

Ni in the amount of 2.0% is used as the hydrogenating component. The process is implemented at the temperature of 220° C., pressure of 1.5 MPa, molar ratio H₂:hydrocarbon of 3.0:1, and feed space velocity of 1.0 h⁻¹.

Depth of n-butane isomerization into isobutane after 10, 200 hours and after regeneration of the catalyst is given in Table 2.

### Example 13

C₅-C₆ cut is used as the feedstock. The process is implemented on a pilot plant at the temperature of 180° C., pressure of 4.0 MPa, molar ratio H₂:hydrocarbon of 3.0:1, and feed space velocity of 1.0 h⁻¹ on a catalyst with the average pore diameter of 20 nm, which has the following composition, weight %:

| | |
|---|---|
| Zirconium oxide | 70.98 |
| Aluminum oxide | 13.00 |
| Titanium oxide | 1.09 |
| Manganese oxide | 0.95 |
| Iron oxide | 1.68 |
| Sulfuric acid ion SO₄²⁻ | 12.00 |

Pd in the amount of 0.3% is used as the hydrogenating component.

Composition of the feedstock for C₅-C₆ cut isomerization is given in Table 1.

Depth of isomerization for C₅-C₆ cut after 10, 200 hours and after regeneration of the catalyst is given in Table 2.

### Example 14

Isomerization is performed according to the method of example 13 differing in that:
The process is implemented on a catalyst with the average pore diameter of 20 nm, which has the following composition, weight %:

| | |
|---|---|
| Zirconium oxide | 86.00 |
| Aluminum oxide | 10.00 |
| Titanium oxide | 0.30 |
| Manganese oxide | 0.45 |
| Iron oxide | 0.15 |
| Sulfuric acid ion SO₄²⁻ | 3.00 |

Pt in the amount of 0.1% is used as the hydrogenating component. The process is implemented at the temperature of 160° C., pressure of 5.0 MPa, molar ratio H₂:hydrocarbon of 3.0:1, and feed space velocity of 1.5 h⁻¹.

Depth of isomerization for C₅-C₆ cut after 10, 200 hours and after regeneration of the catalyst is given in Table 2.

### Example 15

Isomerization is performed according to the method of example 13 differing in that:
The process is implemented on a catalyst with the average pore diameter of 8 nm, which has the following composition, weight %:

| | |
|---|---|
| Zirconium oxide | 63.40 |
| Aluminum oxide | 19.00 |
| Titanium oxide | 1.90 |
| Manganese oxide | 1.60 |
| Iron oxide | 1.90 |
| Sulfuric acid ion SO₄²⁻ | 12.00 |

Pt in the amount of 0.2% is used as the hydrogenating component. The process is implemented at the temperature of 100° C., pressure of 3.0 MPa, molar ratio H₂:hydrocarbon of 2.0:1, and feed space velocity of 0.5 h⁻¹.

Depth of isomerization for C₅-C₆ cut after 10, 200 hours and after regeneration of the catalyst is given in Table 2.

### Example 16

Isomerization is performed according to the method of example 13 differing in that:
The process is implemented on a catalyst with the average pore diameter of 22 nm, which has the following composition, weight %:

| | |
|---|---|
| Zirconium oxide | 66.35 |
| Aluminum oxide | 18.00 |
| Titanium oxide | 1.00 |
| Manganese oxide | 1.05 |
| Iron oxide | 1.20 |
| Sulfuric acid ion SO₄²⁻ | 12.00 |

Pt in the amount of 0.4% is used as the hydrogenating component. The process is implemented at the temperature of 200° C., pressure of 3.0 MPa, molar ratio H₂:hydrocarbon of 1.0:1, and feed space velocity of 6.0 h⁻¹.

Depth of isomerization for C₅-C₆ cut after 10, 200 hours and after regeneration of the catalyst is given in Table 2.

### Example 17

### Comparative

Isomerization is performed according to the method of example 13 differing in that:
The process is implemented on a catalyst with the average pore diameter of 7 nm, which has the following composition, weight %:

| | |
|---|---|
| Zirconium oxide | 71.53 |
| Aluminum oxide | 14.00 |
| Titanium oxide | 0.08 |
| Manganese oxide | 0.09 |
| Iron oxide | 2.00 |
| Sulfuric acid ion SO₄²⁻ | 12.00 |

Pd in the amount of 0.3% is used as the hydrogenating component. The process is implemented at the temperature of 180° C., pressure of 4.0 MPa, molar ratio H₂:hydrocarbon of 3.0:1, and feed space velocity of 1.0 h⁻¹.

Depth of isomerization for C₅-C₆ cut after 10, 200 hours and after regeneration of the catalyst is given in Table 2.

### Example 18

### Comparative

Isomerization is performed according to the method of example 14 differing in that:
The process is implemented on a catalyst with the average pore diameter of 26 nm, which has the following composition, weight %:

| | |
|---|---|
| Zirconium oxide | 70.98 |
| Aluminum oxide | 15.00 |
| Titanium oxide | 0.05 |
| Manganese oxide | 0.07 |
| Iron oxide | 1.80 |
| Sulfuric acid ion SO₄²⁻ | 12.00 |

Pt in the amount of 0.1% is used as the hydrogenating component. The process is implemented at the temperature of 160° C., pressure of 5.0 MPa, molar ratio H₂:hydrocarbon of 3.0:1, and feed space velocity of 1.5 h⁻¹.

Depth of isomerization for C₅-C₆ cut after 10, 200 hours and after regeneration of the catalyst is given in Table 2.

### Example 19

### Comparative

Isomerization is performed according to the method of example 15 differing in that:
The process is implemented on a catalyst with the average pore diameter of 7 nm, which has the following composition, weight %:

| | |
|---|---|
| Zirconium oxide | 72.70 |
| Aluminum oxide | 14.00 |
| Titanium oxide | 0.09 |
| Manganese oxide | 0.08 |
| Iron oxide | 0.93 |
| Sulfuric acid ion SO₄²⁻ | 12.00 |

Pt in the amount of 0.2% is used as the hydrogenating component. The process is implemented at the temperature of 100° C., pressure of 3.0 MPa, molar ratio H₂:hydrocarbon of 2.0:1, and feed space velocity of 0.5 h⁻¹.

Depth of isomerization for C₅-C₆ cut after 10, 200 hours and after regeneration of the catalyst is given in Table 2.

### Example 20

### Comparative

Isomerization is performed according to the method of example 16 differing in that:
The process is implemented on a catalyst with the average pore diameter of 26 nm, which has the following composition, weight %:

| | |
|---|---|
| Zirconium oxide | 68.65 |
| Aluminum oxide | 16.00 |
| Titanium oxide | 1.12 |
| Manganese oxide | 0.98 |
| Iron oxide | 0.85 |
| Sulfuric acid ion SO₄²⁻ | 12.00 |

Pt in the amount of 0.4% is used as the hydrogenating component. The process is implemented at the temperature of 200° C., pressure of 3.0 MPa, molar ratio H₂:hydrocarbon of 1.0:1, and feed space velocity of 6.0 h⁻¹.

Depth of isomerization for C₅-C₆ cut after 10, 200 hours and after regeneration of the catalyst is given in Table 2.

### Example 21

C₇ cut is used as the feedstock. The process is implemented on a pilot plant at the temperature of 250° C., pressure of 4.0 MPa, molar ratio H₂:hydrocarbon of 5.0:1, and feed space velocity of 0.5 h⁻¹ on a catalyst with the average pore diameter of 8 nm, which has the following composition, weight %:

| | |
|---|---|
| Zirconium oxide | 70.36 |
| Aluminum oxide | 13.00 |
| Titanium oxide | 0.06 |
| Manganese oxide | 0.08 |
| Iron oxide | 1.00 |
| Tungstate ion WO₃²⁻ | 15.00 |

Pt in the amount of 0.5% is used as the hydrogenating component.

Composition of the feedstock for isomerization of C₇ cut is given in Table 2.

Depth of isomerization for C₇ cut after 10, 200 hours and after regeneration of the catalyst is given in Table 2.

### Example 22

Isomerization is performed according to the method of example 21 differing in that:
The process is implemented on a catalyst with the average pore diameter of 20 nm, which has the following composition, weight %:

| | |
|---|---|
| Zirconium oxide | 72.85 |
| Aluminum oxide | 14.00 |
| Titanium oxide | 0.40 |
| Manganese oxide | 0.50 |
| Iron oxide | 0.05 |
| Tungstate ion WO₃²⁻ | 12.00 |

Pt in the amount of 0.2% is used as the hydrogenating component. The process is implemented at the temperature of 160° C., pressure of 3.0 MPa, molar ratio H₂:hydrocarbon of 2.0:1, and feed space velocity of 1.0 h⁻¹.

Depth of isomerization for C₇ cut after 10, 200 hours and after regeneration of the catalyst is given in Table 2.

### Example 23

### Comparative

Isomerization is performed according to the method of example 21 differing in that:
The process is implemented on a catalyst with the average pore diameter of 7 nm, which has the following composition, weight %:

| | |
|---|---|
| Zirconium oxide | 66.35 |
| Aluminum oxide | 13.00 |
| Titanium oxide | 1.80 |
| Manganese oxide | 2.00 |
| Iron oxide | 1.35 |
| Tungstate ion WO₃²⁻ | 15.00 |

Pt in the amount of 0.5% is used as the hydrogenating component. The process is implemented at the temperature of 250° C., pressure of 4.0 MPa, molar ratio H₂:hydrocarbon of 5.0:1, and feed space velocity of 0.5 h⁻¹

Depth of isomerization for C₇ cut after 10, 200 hours and after regeneration of the catalyst is given in Table 2.

### Example 24

### Comparative

Isomerization is performed according to the method of example 22 differing in that:
The process is implemented on a catalyst with the average pore diameter of 26 nm, which has the following composition, weight %:

| | |
|---|---|
| Zirconium oxide | 70.67 |
| Aluminum oxide | 14.00 |
| Titanium oxide | 1.16 |
| Manganese oxide | 0.95 |
| Iron oxide | 1.02 |
| Tungstate ion WO₃²⁻ | 12.00 |

Pt in the amount of 0.2% is used as the hydrogenating component. The process is implemented at the temperature of 160° C., pressure of 3.0 MPa, molar ratio H₂:hydrocarbon of 2.0:1, and feed space velocity of 1.0 h⁻¹.

Depth of isomerization for C₇ cut after 10, 200 hours and after regeneration of the catalyst is given in Table 2.

### Example 25

### Similar

Isomerization is performed according to the method of example 21 differing in that:
The process is implemented on a catalyst with the average pore diameter of 3 nm, produced by the method described in the U.S. Pat. No. 6,495,733 B01 J 27/053 Superacid catalyst for hydroisomerization of n-paraffins.

Depth of isomerization for C₇ cut after 10, 200 hours and after regeneration of the catalyst is given in Table 2.

Parameters of the isomerization process as per examples 1-24 (isomerization depth), average pore diameter for the catalyst, and its chemical composition are given in Table 2.

The conducted experiments indicate that it is necessary to use a zirconium oxide catalyst with the average pore diameter of 8-24 nm to ensure the efficient isomerization of C₄-C₇ hydrocarbons. Both deep isomerization and maintenance of the isomerization depth for the entire life cycle and after regeneration performed after the catalyst coking is ensured in this case.

When C₄-C₇ hydrocarbons are isomerized using a zirconium oxide catalyst with the average pore diameter below 8 nm (Examples 7, 9, 11, 17, 19, and 23), then the isomerization depth is reduced already after 200 hours and not recovered completely after regeneration.

When using a zirconium oxide catalyst with the average pore diameter over 24 nm for the isomerization process (Examples 8, 10, 12, 18, 20, and 24), both the initial and the final depth of isomerization for C₄-C₇ paraffin hydrocarbons is reduced by 10-20% relatively.

TABLE 1
Feedstock composition

| | | |
|---|---|---|
| n-butane | C5-C6 cut | C7 cut |

Composition, weight %.

| | | | |
|---|---|---|---|
| propane | 1.0 | 0.7 | |
| isobutane | | 4.49 | |
| n-butane | 96.0 | 13.11 | |
| isopetane | 3.0 | 25.67 | |
| n-pentane | | 15.92 | |
| 1-pentene | | 0.35 | |
| cyclopentane | | 0.35 | |
| 2,2-dimethylbutane | | 2.24 | |
| 2,3-methylbutane | | 2.31 | |
| 2-methylpentane | | 11.43 | |
| 3-methylpentane | | 8.84 | |
| n-hexane | | 9.60 | 0.01 |
| methylcyclopentane | | 1.14 | 0.09 |
| cyclohexane | | | 0.27 |
| 1,1-dimethylcyclopentane | | | 4.81 |
| benzene | | 4.00 | 4.16 |
| 2,2-dimethylpentane | | 0.19 | 2.72 |
| 2,4-dimethylpentane | | 0.20 | 3.50 |
| 2,2,3-trimethylbutane | | | 0.40 |
| 3,3-dimethylpentane | | | 3.08 |
| 2-methylhexane | | | 23.96 |
| 2,3-dimethylpentane | | | 8.40 |
| 3-methylhexane | | | 29.22 |
| 3-ethylpentane | | | 2.81 |
| n-heptane | | | 15.57 |
| methylcyclohexane | | | 0.23 |
| ethylcyclopentane | | | 0.01 |
| toluene | | | 0.75 |
| Sulfur content, ppm | 5 | 1 | 1 |
| H₂O content, ppm | 3 | 5 | 3 |

TABLE 2
Depth of isomerization for C₄-C₇ hydrocarbons with respect to the catalyst pore diameter

| | Catalyst composition, weight % | | | | | | | | | | | | | |
|---|---|---|---|---|---|---|---|---|---|---|---|---|---|---|
| | | | | | | | Mass ratio of the components in the carrier | | | | | | | Dia. of |
| Example No. | Pt | Pd | Ni | Zn | Ga | Carrier | ZrO₂ | A1₂O₃ | TiO₂ | MnO | Fe₂O₃ | SO₄²⁻ | WO₄²⁻ | pores, nm |
| 1 | | | | | 1.00 | 99.00 | 71.81 | 15.00 | 0.05 | 0.05 | 0.09 | 12.00 | | 8 |
| 2 | | | | | 3.00 | 97.00 | 63.91 | 28.00 | 1.00 | 0.90 | 0.19 | 3.00 | | 24 |
| 3 | | | | 1.20 | | 98.80 | 60.00 | 16.00 | 0.10 | 0.70 | 2.00 | 20.00 | | 22 |
| 4 | | | | 2.80 | | 97.20 | 63.66 | 22.00 | 1.50 | 1.50 | 0.54 | 8.00 | | 20 |
| 5 | | | 1.40 | | | 98.60 | 63.55 | 18.00 | 2.00 | 1.90 | 1.15 | 12.00 | | 20 |
| 6 | | | 2.50 | | | 97.50 | 64.48 | 17.00 | 1.40 | 1.60 | 1.02 | 12.00 | | 20 |
| 7 comp | | | | | 1.20 | 98.80 | 61.75 | 26.00 | 0.05 | 0.05 | 0.95 | 10.00 | | 7 |
| 8 comp. | | | | | 2.80 | 97.20 | 58.90 | 30.00 | 1.00 | 1.00 | 1.30 | 5.00 | | 26 |
| 9 comp. | | | | 1.30 | | 98.70 | 63.65 | 12.00 | 1.15 | 0.40 | 1.50 | 20.00 | | 7 |
| 10 comp. | | | | 2.60 | | 97.40 | 66.00 | 10.00 | 1.00 | 1.20 | 1.20 | 18.00 | | 26 |
| 11 comp. | | | 1.50 | | | 98.50 | 67.40 | 15.00 | 1.50 | 1.40 | 1.20 | 12.00 | | 7 |
| 12 comp. | | | 2.00 | | | 98.00 | 66.84 | 18.00 | 0.07 | 0.09 | 1.00 | 12.00 | | 26 |
| 13 | | 0.30 | | | | 99.70 | 70.98 | 13.00 | 1.09 | 0.95 | 1.68 | 12.00 | | 20 |
| 14 | 0.10 | | | | | 99.90 | 86.00 | 10.00 | 0.30 | 0.45 | 0.15 | 3.00 | | 20 |
| 15 | 0.20 | | | | | 99.80 | 63.40 | 19.00 | 1.90 | 1.60 | 1.90 | 12.00 | | 8 |
| 16 | 0.40 | | | | | 99.60 | 66.35 | 18.00 | 1.00 | 1.05 | 1.20 | 12.00 | | 22 |
| 17 comp. | | 0.30 | | | | 99.70 | 71.53 | 14.00 | 0.08 | 0.09 | 2.00 | 12.00 | | 7 |
| 18 comp. | 0.10 | | | | | 99.90 | 70.98 | 15.00 | 0.05 | 0.07 | 1.80 | 12.00 | | 26 |
| 19 comp. | 0.20 | | | | | 99.80 | 72.70 | 14.00 | 0.09 | 0.08 | 0.93 | 12.00 | | 7 |
| 20 comp. | 0.40 | | | | | 99.60 | 68.65 | 16.00 | 1.12 | 0.98 | 0.85 | 12.00 | | 26 |
| 21 | 0.50 | | | | | 99.50 | 70.36 | 13.00 | 0.06 | 0.08 | 1.00 | | 15.00 | 8 |
| 22 | 0.20 | | | | | 99.80 | 72.85 | 14.00 | 0.40 | 0.50 | 0.05 | | 12.00 | 20 |
| 23 comp. | 0.50 | | | | | 99.50 | 66.35 | 13.00 | 1.80 | 2.00 | 1.35 | | 15.00 | 7 |
| 24 comp. | 0.20 | | | | | 99.80 | 70.67 | 14.00 | 1.16 | 0.95 | 1.02 | | 12.00 | 26 |
| 25 similar | | | | | | | | | | | | | | 3 |

| | Isomerization depth | | | | | | | | |
|---|---|---|---|---|---|---|---|---|---|
| | n-butane | | | C5-C6 cut | | | C7 cut | | |
| Example No. | 10 h | 200 h | After regeneration | 10 h | 200 h | After regeneration | 10 h | 200 h | After regeneration |
| 1 | 50 | 50 | 50 | | | | | | |
| 2 | 52 | 52 | 52 | | | | | | |
| 3 | 48 | 48 | 48 | | | | | | |
| 4 | 50 | 50 | 50 | | | | | | |
| 5 | 46 | 46 | 46 | | | | | | |
| 6 | 48 | 48 | 48 | | | | | | |
| 7 comp | 50 | 46 | 44 | | | | | | |
| 8 comp. | 38 | 38 | 38 | | | | | | |
| 9 comp. | 46 | 44 | 43 | | | | | | |
| 10 comp. | 43 | 40 | 41 | | | | | | |
| 11 comp. | 44 | 40 | 40 | | | | | | |
| 12 comp. | 41 | 38 | 39 | | | | | | |
| 13 | | | | 28 | 28 | 28 | | | |
| 14 | | | | 30 | 30 | 30 | | | |
| 15 | | | | 30.5 | 30.0 | 30.5 | | | |
| 16 | | | | 31 | 31 | 31 | | | |
| 17 comp. | | | | 28 | 24 | 26 | | | |
| 18 comp. | | | | 22 | 22 | 22 | | | |
| 19 comp. | | | | 35 | 30 | 32 | | | |
| 20 comp. | | | | 28 | 26 | 28 | | | |
| 21 | | | | | | | 35 | 35 | 35 |
| 22 | | | | | | | 36 | 36 | 36 |
| 23 comp. | | | | | | | 32 | 29 | 30 |
| 24 comp. | | | | | | | 32 | 30 | 31 |
| 25 similar | | | | | | | 30 | 27 | 28 |

1. A method comprising:
isomerizing paraffin hydrocarbons C₄-C₇ in a hydrogen atmosphere at a temperature selected from a range of about 100° C. to about 250° C., at a pressure selected from a range of about 1.0 MPa to about 5.0 MPa, at a feed space velocity selected from a range of about 0.5 h⁻¹ to about 6.0 h⁻¹, and with a molar ratio of hydrogen to hydrocarbons ranging from about 0.1:1 to about 5:1, the isomerizing step occurring in the presence of a porous zirconium oxide catalyst having an average pore diameter ranging from about 8 nm to about 24 nm to maintain constant isomerization depth in operation and after oxidative regeneration; and
stabilizing products of isomerization and/or fractioning the products of isomerization to recover individual hydrocarbons or high-octane fractions.

2. The method of claim 1, wherein a composition of the zirconium oxide catalyst
comprises, by weight %:
97.00-99.90 of a carrier, the carrier comprising:

| | |
|---|---|
| zirconium oxide | 60.00-86.00 |
| aluminum oxide | 10.00-30.00 |
| titanium oxide | 0.05-2.00 |
| manganese oxide | 0.05-2.00 |
| iron oxide | 0.05-2.00 |
| SO₄²⁻ or WO₃²⁻ | 3.00-20.00 |

hydrogenating component 0.10-3.00, the hydrogenating component is selected from the group consisting of Pt, Pd, Ni, Zn, Ga and combinations thereof.
